(19) United States
(12) Patent Application Publication
(10) Pub. No.: US 2026/0311350 A1
(43) Pub. Date: Oct. 8, 2026

(54) SYSTEM, METHOD AND COMPUTER-ACCESSIBLE MEDIUM FOR PROVIDING AUTOMATED IMAGE PATH REGISTRATION

(71) Applicant: KONINKLIJKE PHILPS N.V., Eindhoven (NL)

(72) Inventors: Dave Vader, Concord, MA (US); James Mclean, Somerville, MA (US); Jason Tucker-Schwartz, Melrose, MA (US); Eman Namati, Concord, MA (US); Damon Depaoli, Ottawa (CA); Ying Mu, Bedford, MA (US); Daniel Gourdeau, Medford, MA (US); Vivek Walimbe, Westford, MA (US)

(21) Appl. No.: 19/483,437

(22) PCT Filed: May 15, 2024
(86) PCT No.: PCT/US2024/029404

§ 371 Date: Nov. 11, 2025

Related U.S. Application Data
(60) Provisional application No. 63/466,551, filed on May 15, 2023.

(51) Int. Cl. G06T 7/33; A61B 6/46; A61B 6/50; A61B 34/20; G06T 7/00; G06V 10/26; G06V 10/82

(52) U.S. Cl. CPC G06T 7/337; A61B 6/463; A61B 6/503; A61B 6/504; A61B 34/20; G06T 7/0014; G06V 10/26; G06V 10/82; G06T 2207/10048; G06T 2207/10056; G06T 2207/10064; G06T 2207/10101; G06T 2207/10132; G06T 2207/20081; G06T 2207/20084; G06T 2207/30096; G06T 2207/30101; G06T 2207/30204; G06V 2201/03

(57) ABSTRACT

Exemplary methods and systems can be provided for obtaining information regarding at least one feature of an object. For example, it is possible to acquire an intravascular dataset and an extravascular dataset, automatically detect a marker within each of at least two images of the extravascular dataset, automatically register the feature(s) in the images, automatically generate an imaging path based on at least one position of the marker within at least one of the images based on the registered feature, automatically register the intravascular dataset to the imaging path to generate the registered intravascular data, and automatically overlay a representation of the registered intravascular data within at least one of the images.

### CROSS REFERENCE TO RELATED APPLICATION(S)

This application relates to and claims the benefit of priority from U.S. Provisional Patent Application No. 63/466,551, filed on May 15, 2023, the entire disclosure of which is incorporated herein by reference in its entirety.

### FIELD OF THE DISCLOSURE

The present disclosure relates generally to the technology of electromagnetic radiation used for medical imaging, and more particularly, to system, apparatus, and method for detecting and displaying an imaging path.

### BACKGROUND INFORMATION

Electromagnetic radiation is a useful tool to image biological structures. When imaging a cavity within a biological structure, a probe is often used to deliver the electromagnetic radiation and recover an internal image. It can be difficult to assess where the internal image has been taken using the probe, relative to an external view of the structure. Imaging using electromagnetic radiation capable of traversing through an entire biological structure, from an external position, can help to deduce the relative position of an imaging probe, but still, it remains difficult to assess this position in the presence of motion from either the structure or the probe.

Accordingly, there is a need to provide apparatuses, systems and/or methods to improve the localization of internal imaging probes and to present the information in the presence of motion, and address at least some of the deficiency with the prior systems and methods.

### SUMMARY OF EXEMPLARY EMBODIMENTS

Such issues and/or deficiencies can at least be partially addressed and/or overcome by providing system, apparatus and method for automatically locating and registering internal probe locations within a set of external structure images, by providing exemplary embodiments according to the present disclosure.

According to an exemplary embodiment of the present disclosure, exemplary apparatuses, systems, and methods for automatically locating (e.g., detecting) and registering internal probe locations (e.g., the distal tip of the probe, e.g., within a catheter, within a cardiac catheter) and corresponding images (e.g., images of a lumen, e.g., images of a guide-wire, e.g., images of an artery wall) & automatically detected structures (e.g., biomarkers, e.g., arterial plaques, e.g., side-branches, e.g., intravascular devices) as well as the probe's path of motion (e.g., during the placement of a probe, e.g., during an imaging pullback of the probe), within a set of external structure images (e.g., angiography images, fluoroscopy images, X-ray images, e.g., ultrasound images, e.g., magnetic resonance images) can be provided.

During cardiac catheterization, X-ray imaging can be performed to diagnose (e.g., diagnostic angiogram) and plan and guide treatment (e.g., stenting, a ballooning, plaque modification). In some exemplary cases, intravascular sensing (e.g., imaging, e.g., physiology) can improve procedural outcomes by providing localized diagnostic and prognostic information not readily available on the X-ray angiogram, for example the size of the lumen (e.g., at high resolution), the position and diameter of the external elastic laminae (EEL), the composition and location of a plaque (e.g., a lipid plaque or a calcified plaque). Intravascular imaging devices often image in multiple axes, performing a rotation (e.g., around the vessel circumference) and a translation (e.g., a pullback of the imaging core along the longitudinal direction of the vessel) to provide a 2-dimensional (2D) or a 3-dimensional (3D) (e.g., if the modality provides, for example a depth axis) map of the intraluminal structure.

When intravascular imaging is performed, it can be complicated to understand where the internal information has originated with respect to the X-ray angiogram, due to the motion of both the intravascular imaging probe during an imaging and due to the patient motion (e.g., heartbeat, respiratory, bodily movements). The challenge to register the information is compounded by modern intravascular imaging system capabilities, imaging much larger areas (e.g., 360° rotation and greater than about 100 mm translation) and at much faster speeds than traditional concepts (e.g., 50 mm/s) while X-ray systems provide relatively low imaging speeds (e.g., 15 fps). Furthermore, the cardiac catheterization laboratory is a fast-paced, life or death environment that requires efficacy and efficiency providing little opportunity for a physician to exit the sterile area & provide manual inputs to a device for registration.

Traditional methods to register the imaging path of the intravascular imaging device onto an extravascular image (e.g., an angiogram) required user input, were performed on a single extravascular image, removing the complexities of motion but minimizing the value of the temporal extravascular image sequence, showing multiple views of the heart as it beats. Furthermore, traditional methods are able to perform registration solely on contrast injected coronary angiograms. The apparatuses, systems, and methods according to the exemplary embodiments of the present disclosure can overcome these shortcomings and maximizes the potential a combined intravascular and extravascular imaging approach to care, and improves patient outcomes.

In an exemplary embodiment of the present disclosure, a method and an intravascular imaging device can be provided which can be used to receive an intravascular image set and an extravascular image set, automatically detect the beginning and end of an intravascular imaging translation within the extravascular image set, and overlay the imaging path of the intravascular imaging translation, and/or other intravascular information, onto an image or several images within the extravascular image set. The exemplary system and method can be used to register the imaging path automatically, with no user input, overlays the intravascular imaging path on multiple extravascular images, overcoming the motion of both the imaging probe and the patient, and perform an exemplary registration, e.g., without the presence of contrast filling the coronary vessels.

According to another exemplary embodiment of the present disclosure, methods and systems can be provided for obtaining information regarding at least one feature of an object. For example, it is possible to acquire an intravascular dataset and an extravascular dataset, automatically receive information regarding the feature(s) within the extravascular dataset, automatically generate an imaging path based on the received information regarding the feature(s); automatically register the intravascular dataset to the imaging path to generate the registered intravascular data, and automatically overlay a representation of the registered intravascular data within at least one image of the extravascular dataset.

For example, the feature(s) can includes a radiopaque marker on an intravascular probe. The registered intravascular dataset can include at least one intravascular sensing dataset. The intravascular sensing dataset can include data regarding at least one of a pressure, an electric field, or flow. Additionally or alternatively, the registered intravascular dataset can include at least one intravascular imaging dataset, at least one of optical coherence tomography (OCT) data, intravascular ultrasound (IVUS) data, Near-infrared spectroscopy (NIRS) data, reflectance data, and/or Fluorescence Lifetime Imaging Microscopy (FLIM) data. The extravascular dataset can include data for coronary angiography images.

In another exemplary embodiment of the present disclosure, the overlayed representation can indicate a location of a stenosis. The overlayed representation can further indicate a type of plaque at the stenosis. The overlayed representation can indicate a measurement output at a specific location along a coronary vessel. The measurement output can comprise a plaque burden, a lipid burden, and/or a calcium burden. The automatically detecting the feature can comprise executing a machine learned procedure for an object segmentation (e.g., U-Net, Res-Net, etc.). The automatically detecting the feature can comprise executing a machine learned procedure for an object detection. The automatically generating the image path can comprise automatically interpolating datapoints.

According to further exemplary embodiments of the present disclosure, it is possible to register the image path between extravascular images within the extravascular dataset. The automatically registering the image path can comprise executing a machine learned non-rigid transformation procedure between the images within the extravascular dataset. In yet further exemplary embodiment of the present disclosure, it is possible to automatically segment a coronary vasculature within the extravascular dataset. Additionally or alternatively, it is possible to segment data associated with a guide wire within the extravascular dataset. The intravascular dataset can be a multimodal intravascular imaging and spectroscopy dataset.

According yet further exemplary embodiment of the present disclosure, methods and systems for obtaining information regarding at least one feature of an object. For example, it is possible to acquire an intravascular dataset and an extravascular dataset, automatically detect a marker within each of at least two images of the extravascular dataset, automatically register the feature(s) in the images, automatically generate an imaging path based on at least one position of the marker within at least one of the images based on the registered feature, automatically register the intravascular dataset to the imaging path to generate the registered intravascular data, and automatically overlay a representation of the registered intravascular data within at least one of the images.

In one exemplary embodiment of the present disclosure, the feature(s) can include at least one portion of a guidewire, an imaging catheter, a guide catheter and/or an intravascular device. Alternatively or in addition, the feature(s) can include at least one portion of a coronary vessel, at least partially contrast-filled coronary artery, and/or a coronary feature in absence of contrast. The automatically registering the intravascular dataset to the imaging path can comprise automatically addressing a vessel foreshortening in the extravascular images. The automatically addressing can use a constrained interpolation. The acquisition of the intravascular dataset and the extravascular dataset can be synchronized with one another. In addition or alternatively, the automatically registering the intravascular dataset to the imaging path can comprise automatically addressing at least one temporal offset in the acquisition of the extravascular dataset and the intravascular dataset. The temporal offset(s) can be characterized a priori to the acquisition of the intravascular data.

According to yet additional exemplary embodiment of the present disclosure, the representation of the intravascular data can be registered and displayed on more than one of the images from the extravascular dataset and/or on any image from the extravascular dataset thereby achieving an imaged derived threshold. The imaged derived threshold can include a registration confidence threshold and/or an image quality threshold. In addition or alternatively, the representation of the intravascular data can be registered and displayed on an automatically selected image from the extravascular dataset and/or on an enhanced image from the extravascular dataset. The registering of the intravascular dataset can comprise automatically colocalizing automatically detected side branches in the intravascular dataset and the extravascular dataset.

In still further exemplary embodiments of the present disclosure, the automatically generating the image path comprises interpolating datapoints in a registered space provided by the at least one registered feature. The registering of the intravascular dataset to the imaging path can comprise automatically colocalizing automatically segmented lumen profiles in the intravascular dataset and the extravascular dataset. In addition or alternatively, the registering of the intravascular dataset to the imaging path can comprise automatically colocalizing automatically detected stenosis positions in the intravascular dataset and the extravascular dataset. The automatically detecting of the feature(s) can comprise executing a machine learned procedure for at least one of an object segmentation or an object detection. The automatically registering of the at least one feature can comprise executing a machine learned non-rigid transformation procedure.

The overlayed representation can indicate an automatically detected vascular characteristic and/or a measured vascular characteristic from the intravascular dataset at a specific location along a coronary vessel. The marker can indicate the sensing position of an intravascular probe. The intravascular dataset can be an intravascular sensing dataset and/or an intravascular imaging dataset. The extravascular dataset can be a set of coronary angiography images. The overlayed representation can indicate a location of an automatically detected stenosis, the type of plaque at the stenosis, and/or a lesion with an index of ischemia surpassing a predetermined threshold. The index of ischemia can be an at least partially image-derived index. The marker can be a radiopaque marker.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a flow diagram of a method/process for automatically registering intravascular information on an extravascular image set according to exemplary embodiments of the present disclosure;

FIG. 2 is a set of exemplary graphs illustrating exemplary methods/processes, systems and apparatus for registering the intravascular information on the extravascular image set according to exemplary embodiments of the present disclosure;

FIG. 3 is a set of illustrations using extravascular image Segmentation. In some embodiments, bodily objects (e.g., contrast filled vasculature), foreign objects (e.g., a guide catheter, e.g., a guide wire) or both, are detected and/or segmented to guide a registration of the multiple extravascular images to one another or to guide the registration of extravascular images to intravascular images.

FIG. 4 is a set of illustrations for an extravascular object detection according to exemplary embodiments of the present disclosure;

FIG. 5 is a set of illustrations for a Non-Rigid Registration/Creating the image deformation map to overcome motion during an imaging session & create an accurate imaging path, according to exemplary embodiments of the present disclosure;

FIG. 6 is a set of illustrations providing examples of Intravascular to extravascular registration refinement using detected side branch positions within the extravascular images and the intravascular images, according to exemplary embodiments of the present disclosure;

FIG. 7 is a set of illustrations providing examples of Measurement profiles from the extravascular images overlayed on multiple frames from the extravascular image data set, according to exemplary embodiments of the present disclosure;

FIG. 8 is a set of illustrations providing exemplary Contrast Free Object Detection for Image path reconstruction and registration, according to exemplary embodiments of the present disclosure;

FIG. 9 is a set of illustrations providing exemplary Contrast Free Object segmentation for image path reconstruction and registration, according to another exemplary embodiments of the present disclosure;

FIG. 10 is a set of illustrations providing examples of Intravascular image feature (e.g., automatically detected features) overlayed on an extravascular image with and without contrast agents, according to exemplary embodiments of the present disclosure;

FIG. 11 is a system diagram of an Intravascular imaging system, according to an exemplary embodiment of the present disclosure;

FIG. 12 is an exemplary display of co-registered Intravascular imaging and extravascular imaging on Intravascular imaging with the user interface (UI), using the exemplary system of FIG. 11, according to exemplary embodiments of the present disclosure;

FIG. 13 is an exemplary display of the co-registered Intravascular imaging and extravascular imaging on the Intravascular imaging with the user interface (UI), using the exemplary system of FIG. 11, according to yet other exemplary embodiments of the present disclosure; and

FIG. 14 is an exemplary computer system, according to an exemplary embodiment of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION OF EXAMPLE EMBODIMENTS

The following description of exemplary embodiments provides non-limiting representative examples referencing numerals to particularly describe features and teachings of different aspects of the present disclosure. The embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the invention to such an extent that other implementations, not specifically covered but within the knowledge of a person of skill in the art having read the description of embodiments, would be understood to be consistent with an application of the present disclosure. In some exemplary embodiments of the present disclosure, the external structure images can be provided by an exemplary system (e.g., an X-Ray system, an angiography system, a fluoroscopy system) in accordance with the exemplary embodiments of the present disclosure. The exemplary system can be used, for example, to image a patient or a region of a patient. In some exemplary embodiments of the present disclosure, the exemplary system can be used to perform a coronary angiogram. In some embodiments the exemplary system can image the chest of a patient during a cardiac catheterization. The exemplary images (e.g., X-Ray images) provided by the exemplary system can be used to guide a cardiac catheterization procedure.

In some examples, a radiopaque agent can be injected into the coronary arteries in order to visualize the structure on the exemplary image of a patient's chest. In some embodiments, the exemplary system can perform a coronary angiogram (e.g., while a probe or cardiac catheter is inserted into a coronary vessel). In some exemplary embodiments of the present disclosure, the exemplary system can have a specific frame rate (e.g., 5 fps, 7 fps, 10 fps, 15 fps, 30 fps, 60 fps, etc.), and an image sequence (e.g., a video) can be acquired at the specified frame rate. While an image sequence is being acquired, there can be motion caused by the living specimen (e.g., breathing, heart-beating, bodily movements) or there can be motion caused by the exemplary system being displaced. In some exemplary embodiments of the present disclosure, an image sequence can be taken while an imaging probe is located within a coronary vessel and its position will move over time, due to any motion source. According to further exemplary embodiments of the present disclosure, an image sequence can be taken while a probe is being translated.

According to certain exemplary embodiments of the present disclosure, the probe being imaged can be partially radiopaque (e.g., fully radiopaque) in certain locations (e.g., the entire probe, e.g., the distal tip) in order to facilitate locating the probe position within an externally acquired exemplary (e.g., X-ray) image. For example, a radiopaque marker can be located on the probe near, for example, the distal tip where imaging can be performed. In an exemplary embodiment of the present disclosure, the probe can be housed by a protective sheath (e.g., a plastic sheath) that is partially transparent (e.g., fully transparent) to certain forms of electromagnetic radiation (e.g., visible wavelengths of light and infrared wavelengths of lights and everything in between). The protective sheath can comprise locations with controlled opacity (e.g. radiopacity) (e.g., at specific locations along its length, e.g., at the distal tip of the sheath) in order to facilitate locating the sheath position within an externally acquired X-ray image (e.g., in a set of X-ray images). For example, the probe and its sheath can be comprised or housed within a catheter (e.g., a cardiac catheter). The catheter can be deployed to image intraluminal objects such as, for example, the interior of an artery wall.

According to additional exemplary embodiments of the present disclosure, the catheter can be connected to a catheter interface unit (CIU), and the CIU can be connected to a console device. Such interconnected configuration can make up an exemplary intravascular imaging system. For example, the intravascular imaging system can be used to image the interior of an artery wall using an optical modality (e.g., optical coherence tomography, near-infrared spectroscopy (NIRS), diffuse reflectance, Raman spectroscopy, etc.). In some exemplary embodiments of the present disclosure, the intravascular imaging system can be used to image the interior of an artery wall using an ultrasonic modality (e.g., a combined optical & ultrasonic modality, e.g., photoacoustic modality). In some exemplary embodiments of the present disclosure, the intravascular imaging system can be a multi-modal intravascular imaging system, such as, e.g., an OCT-NIRS imaging system, configured to provide both high resolution intravascular imaging and measurements (e.g., using the OCT features) as well as highly specific & accurate plaque characterization (e.g., using the NIRS features).

According to certain exemplary embodiments of the present disclosure, an intravascular imaging system can be provided which can comprise an imaging probe (e.g. a multimodal imaging probe, an intravascular imaging probe, etc.). For example, the intravascular imaging probe can be designed to be observable within an x-ray image (e.g., using radiopaque substances). The intravascular imaging probe can have a protective sheath and an imaging core, whereas, for example the imaging core is translated to create an image through the protective sheath. The intravascular imaging probe can also comprise at least one radiopaque marker. For example, a radiopaque imaging marker (e.g., an imaging marker) can be located on the imaging core of the imaging probe, so as to be able to monitor any translation of the imaging portion. A radiopaque marker can also reside on the protective sheath portion of an imaging probe, for example to mark a specific position, for example the distal tip of the catheter (e.g., to allow a clinician to the catheter to the correct position in the coronary vasculature). In some exemplary embodiments of the present disclosure, the radiopacity of any component of the imaging probe can be tuned (e.g., optimized) to provide a controlled amount of contrast within an x-ray image. The intravascular imaging probe can be an optical imaging probe (e.g., optical coherence tomography, near-infrared spectroscopy (NIRS), diffuse reflectance, Raman spectroscopy, etc.). In some further exemplary embodiments of the present disclosure, the intravascular imaging probe can be an ultrasonic imaging probe with an ultrasonic modality (e.g., a combined optical and ultrasonic modality, e.g., photoacoustic modality). In some further exemplary embodiments of the present disclosure, the intravascular imaging probe can be a multi-modal intravascular imaging probe, such as, e.g., an OCT-NIRS imaging system, configured to provide both high resolution intravascular imaging and measurements (e.g., using the OCT features) as well as highly specific & accurate plaque characterization (e.g., using the NIRS features). In some further exemplary embodiments of the present disclosure, the intravascular imaging probe can be a multi-modal intravascular imaging probe, such as, e.g., an OCT-IVUS imaging system.

According to further exemplary embodiments of the present disclosure, an intravascular imaging system can be connected to an X-ray system, thus facilitating the logging (e.g., saving, e.g., writing to disk) and processing of X-ray images (e.g., image sequences). For example, logging and/or processing of the X-ray image data can be triggered by a start of an imaging probe pullback (e.g., providing synchronous intravascular images and X-ray images). In some exemplary embodiments of the present disclosure, the exemplary intravascular imaging system can retrieve the X-ray images via a wired connection to an angiography system (e.g., a video-out signal). In some exemplary embodiments of the present disclosure, the exemplary intravascular imaging system can retrieve the X-ray images wirelessly (e.g., over a wireless network, e.g., from a PACS server). The intravascular imaging system can also use the triggered data to automatically register the location of intravascular landmarks on the X-ray image (e.g., on many X-ray images).

According to yet another exemplary embodiment of the present disclosure, an intravascular imaging system calibration configuration can be provided (e.g., to improve temporal registration between intravascular and extravascular imaging systems). System calibration can comprise performing an imaging session of a calibration setup on a specific X-ray system (e.g., acquiring both intravascular imaging system data and extravascular imaging system data, e.g., simultaneously). For example, a system calibration setup can include recording or saving information regarding the X-ray system. Further or alternatively, the exemplary intravascular imaging system can be calibrated for multiple X-ray systems (e.g., wherein a given extravascular system is manually selected or automatically detected after being in communication with the intravascular imaging system. e.g., wherein, there exists a stored calibration file for each extravascular imaging system on the intravascular imaging system, e.g., wherein the calibration files were generated after a calibration procedure on a given extravascular system).

According to certain exemplary embodiments of the present disclosure, the location of an intravascular probe's radiopaque imaging marker can be detected automatically using a procedure (e.g., a machine learned procedure, e.g., a deep learning procedure, e.g., an object detection procedure, e.g., a YOLO procedure) which can facilitate the registration of the intravascular probe's imaging position within an extravascular image (e.g., a fluoroscopy image, e.g., an angiography image). For example, the position of a radiopaque imaging marker from multiple extravascular images (e.g., all images provided by a fluoroscopy or cine acquisition) can be automatically detected and used to automatically infer (e.g., interpolate) the intravascular probes imaging position between the extravascular images (e.g., the registered extravascular images) and therefore facilitate the registration of the imaging path to the intravascular data from the intravascular probe. This exemplary feature can be advantageous, for example, if the intravascular imaging probe's frame-rate, or translation speed, is higher than the extravascular imaging frame-rate. In order to infer an intravascular imaging path across multiple extravascular images in the presence of patient movement (e.g., heart motion, patient motion, extravascular imaging device motion, etc.), the automatically detected features from the multiple extravascular images can utilize an inter-frame registration using a registration procedure (e.g., a rigid transformation procedure, auto-correlation based procedure, a non-rigid transformation procedure, a warping procedure, a machine learned warping procedure, VoxelMorph, etc.).

In some embodiments for the present disclosure, an automatically-detected extravascular image feature (e.g., an automatically detected coronary architecture, automatically segmented guide wire, etc.) can be used to automatically estimate a transformation map (e.g. a deformation map) between extravascular images, in order to perform a registration procedure (e.g., to register the extravascular images, register the extravascular image features, etc.). In some exemplary embodiments of the present disclosure, automatically detected radiopaque markers from several extravascular image (e.g., several imaging timepoints) can include the transformation map applied thereto (e.g., as the radiopaque imaging marker is known to have moved during the acquisition of the extravascular images) in order to track the imaging path of the radiopaque marker on a specific frame (e.g., in a registered space). According to certain exemplary embodiments of the present disclosure, a neural network is trained to detect multiple features of interest in angiography images collected in varying imaging conditions (radiation, flush delivery), system specifications (frame rate, resolution), and biological environments (RCA, LDA, etc, in presence of multiple guidewires, stents, internal radio opaque markers). According to certain exemplary embodiments of the present disclosure, a spatio-temporal neural network (e.g., Recurrent CNN, MeMOTR, etc.) can be trained to detect features of interest (e.g., radiopaque imaging marker, guide catheter, guidewire, coronary architecture, etc.) in varying imaging conditions, system specifications and/or biological environments.

Radiopaque contrast injection into the coronary arteries is a routine procedure that allows a clinician to view the coronary vasculature, for example, to view the degree of a stenotic plaque and diagnose coronary artery disease. Radiopaque contrast agents, however, can be toxic and the toxicity varies based on a patient's condition. For this reason, providing a method of registration between intravascular imaging and extravascular imaging, even in the presence of reduced vessel visibility (e.g., a reduced contrast) on the extravascular image is highly valuable (e.g. to inform stent placement location, e.g., to inform plaque modification location).

In some exemplary embodiments of the present disclosure, an imaging path can be automatically detected based on a moving radiopaque imaging marker registered to an automatically detected foreign object within the extravascular image (e.g., a guide wire, a guide catheter, etc.). An imaging marker can travel variable distances between extravascular images within an extravascular dataset based on patient motion or based on the angle of the 2D extravascular imaging system relative to imaged vessel & imaging path direction (e.g., cause of vessel foreshortening). In some exemplary embodiments of the present disclosure, such detection and registration can be used to generate a virtual vessel foreshortening map to provide a higher accuracy co-registration in the presence of a reduced injected contrast (e.g., reduced vessel architecture visibility, e.g., during a reduced imaging session) or even in the presence of no injected contrast (e.g., no vessel architecture visibility, during a contrast-free imaging session, etc.). In some exemplary embodiments of the present disclosure, an imaging path can be automatically detected (e.g., throughout a set of extravascular images) based on a moving radiopaque imaging marker registered to an estimated path between two foreign object (e.g., a tip of a guide catheter and the tip of a guide wire, etc.), and can utilize a measure of radiopaque imaging marker angle (e.g., in the case that the radiopaque imaging marker is cylindrical). In some exemplary embodiments of the present disclosure, the path of the imaging marker can be automatically detected during a contrast-free imaging session and registered to a previously acquired extravascular image with contrast injected (e.g., a diagnostic angiogram).

In some further exemplary embodiments of the present disclosure, extravascular image quality can be assessed prior to a registration routine or after a registration routine. In some embodiments of the present disclosure, an image quality can be configured to automatically toggle whether an overlay is provided on a given frame. For example, an image quality overlay can be based on, automatically measured extravascular imaging device characteristics (e.g., frame rate, contrast, brightness) or automatically measured imaging motion. In some embodiments of the present disclosure, a registration quality metric (e.g. an index) can be configured to automatically toggle whether an overlay is provided on a given frame. A registration quality metric can be based on, for example, automatically detected speed of the intravascular device pullback, automatically detected curvature of the imaging path, automatically detected consistency of observed X-ray signal for the imaging marker, automatically detected range of detected foreshortening values, automatically detected overlap of the detected pullback path with the detected vessel and guide catheter location, or based on a machine learned confidence metric associated with intermediate and final procedure outputs. In some further exemplary embodiments of the present disclosure, any automatically imaged-derived characteristics within the intravascular or extravascular imaging datasets that reach a pre-determined threshold (e.g., registration confidence threshold, image quality threshold, etc.) can adjust the appearance of an overlay. In yet another exemplary embodiment of the present disclosure, an automatically image-derived characteristic can cause a different set of feature processing methods (e.g., machine learning model, multi-modality image registration, feature tracking, etc.) to be utilized in that case. In yet another exemplary embodiment of the present disclosure, quality metrics on the results of the feature and image processing (e.g., geometric quality of the path, overlap of the detected radiopaque marker path with coronary architecture, estimated level of foreshortening, etc.) can be used to determine whether the overlays are displayed on the extra-vascular or intra-vascular images. In yet another exemplary embodiment of the present disclosure, quality metrics on the results can be used to determine if an additional set of computational methods (e.g., second, third, etc.) can be applied to obtain improved results, and/or to correct existing results.

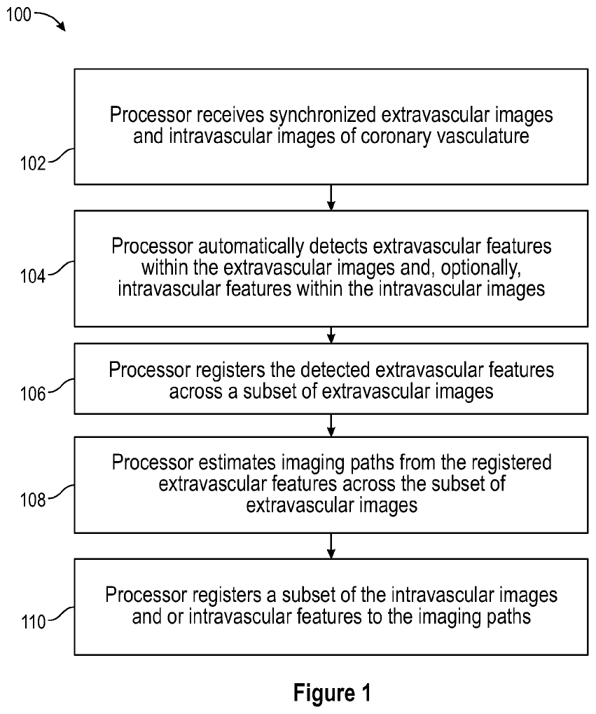

FIG. 1 shows a flow diagram of a method/process 100 for automatically registering intravascular information on an extravascular image set according to exemplary embodiments of the present disclosure. The exemplary method/process shown in FIG. 1 can be executed by the exemplary system and/or computer-accessible medium according to the exemplary embodiments of the present disclosure, and can provided a fully automated image path reconstruction over multiple synchronized images from an extravascular imaging dataset, and an intravascular imaging dataset. For example, as shown in FIG. 1, in procedure 102, the processor can receive synchronized extravascular images (e.g., in the presence of injected contrast media, in the absence of injected contrast media, etc.) and intravascular images of coronary vasculature. In procedure 104, the processor can automatically detect extravascular features within the extravascular images and (optionally) intravascular features within the intravascular images. In procedure 106, the processor can register the detected extravascular features across a subset of extravascular images. In procedure 108, the processor can estimate imaging paths from the registered extravascular features across the subset of extravascular images. Further, in procedure 110, for the subset extravascular of images, the processor can register a subset of the intravascular images and/or intravascular features to the imaging paths.

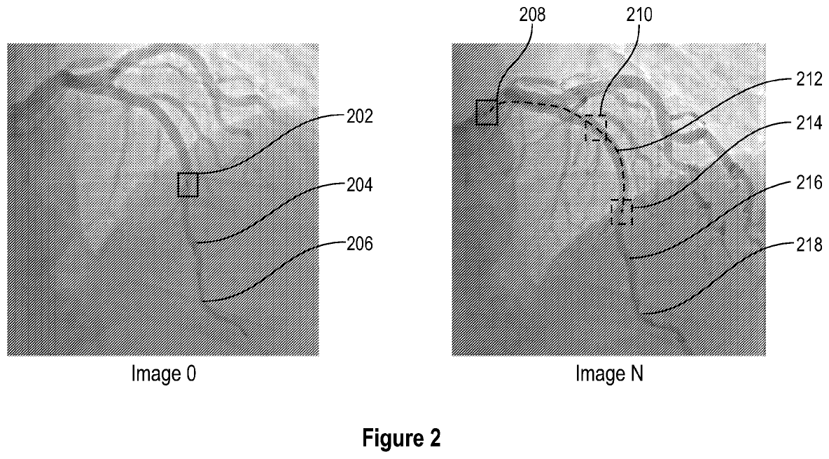

FIG. 2 shows a set of exemplary graphs illustrating exemplary methods/processes, systems and apparatus for registering the intravascular information on the extravascular image set according to exemplary embodiments of the present disclosure. As shown in FIG. 2, a radiopaque marker 202 (Image 0), 208 (Image N) can be detected at the beginning of the pullback within a coronary tree (e.g., a contrast filled coronary tree) within extravascular Image 0 as well as each extravascular image up to extravascular Image N, representing the end of the intravascular probe translation or similarly, the middle of the intravascular probe translation. The radiopaque marker positions across multiple extravascular images can be used to create an imaging path 212.

To create an accurate imaging path, extravascular image registration can be used, in order to interpolate and further register the imaging path to each extravascular image in the presence of motion (e.g., heart motion, respiratory motion, contrast variability). The registration transformation map (e.g., the vector map, the deformation map, etc.) can be automatically inferred for each extravascular image frame in relation to a reference frame or it can be registered to multiple frames, or to any combination of sequential frames that can improve efficiency or performance of the registration procedure. The original radiopaque marker position 214 is shown in FIG. 2 in Image N 214 along with another radiopaque marker position 210 at a timepoint/image taken between Image 0 and Image N. A radiopaque marker 216 located at the distal tip of a cardiac catheter sheath (e.g., a marker that is not translated during an intravascular imaging pullback) and the position of a guide wire tip 218 are also illustrated in FIG. 2, both of which can be used as a landmark for a registration in the absence (e.g., partial absence) of visible vasculature.

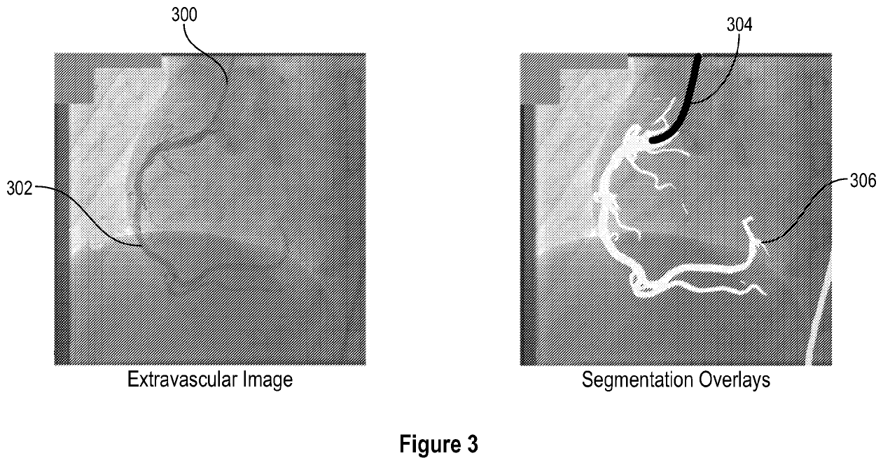

FIG. 3 shows a set of exemplary illustrations using extravascular image segmentation, in accordance with the exemplary embodiments of the present disclosure. For example, bodily objects (e.g., contrast filled vasculature), foreign objects (e.g., a guide catheter, a guide wire, etc.) or both, can be segmented to guide a registration of the multiple extravascular images to one another or to guide the registration of extravascular images to intravascular images. According to certain exemplary embodiments of the present disclosure, the object detection can be performed using certain procedures such as thresholding, morphological operations, Fourier domain analysis, edge detection, etc. or it can be performed using machine learning or deep learning using procedures such as U-net or Res-Net architectures, for example. Turning to the exemplary items identified in FIG. 3, item 300 is a Guide catheter for a coronary intervention/imaging, item 302 is a Contrast-filled artery in extravascular image, item 304 is an Automatically-segmented guide catheter using machine learning/deep learning (e.g., U-Net, ResNet, etc.), and item 306 is an Automatically-segmented coronary vasculature using machine learning/deep learning (U-Net, ResNet, etc.)

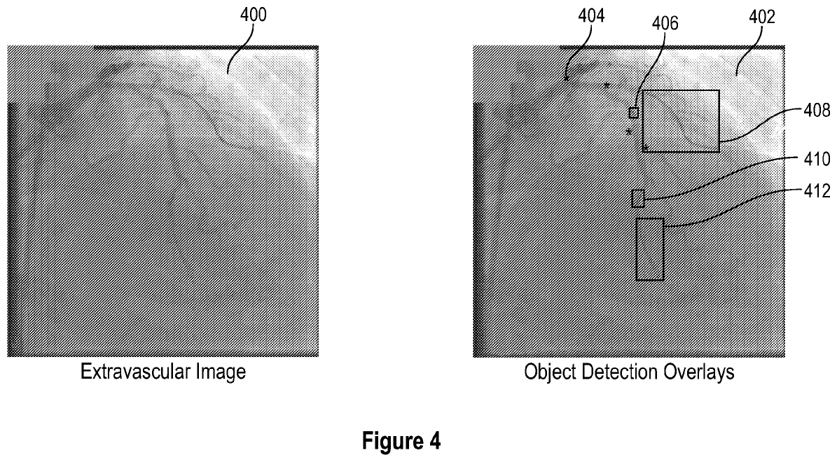

FIG. 4 shows a set of exemplary illustrations for an extravascular object detection according to exemplary embodiments of the present disclosure. According to certain exemplary embodiments of the present disclosure, as illustrate din FIG. 4, bodily objects (e.g., contrast filled vasculature), foreign objects (e.g., a guide catheter tip, e.g., a guide wire edge, e.g., a radiopaque marker) or both, are detected (e.g., localized) to guide a registration of the multiple extravascular images to one another or to guide the registration of extravascular images to intravascular images. The exemplary object detection can be performed using hand-crafted procedures such as thresholding, morphological operations, Fourier domain analysis, edge detection, etc. and/or using machine learning or deep learning using procedures such as YOLO, for example.

For example, the exemplary object detection can be used for a registration inference or registration refinement after an initial registration procedure using semantically segmented objects. Turning to the exemplary items identified in FIG. 4, item 400 is an extravascular image, item 402 is an extravascular image with overlays, item 404 is an automatically-detected side-branch, term 406 is an automatically-detected first radiopaque marker on e.g., intravascular imaging catheter (e.g., for locating the imaging position, a radiopaque housing/can over distal optics-for example, a radiopaque marker that is translated during an intravascular imaging pullback), item 408 is an automatically-detected first guidewire (e.g., within a branch that is not being intravascularly imaged), item 410 is an automatically-detected second radiopaque marker (e.g., intravascular imaging catheter tip position, for example, a radiopaque marker that is not translated during an intravascular imaging pullback), and item 412 is an automatically-detected second guidewire (e.g., within a branch that is being intravascularly imaged).

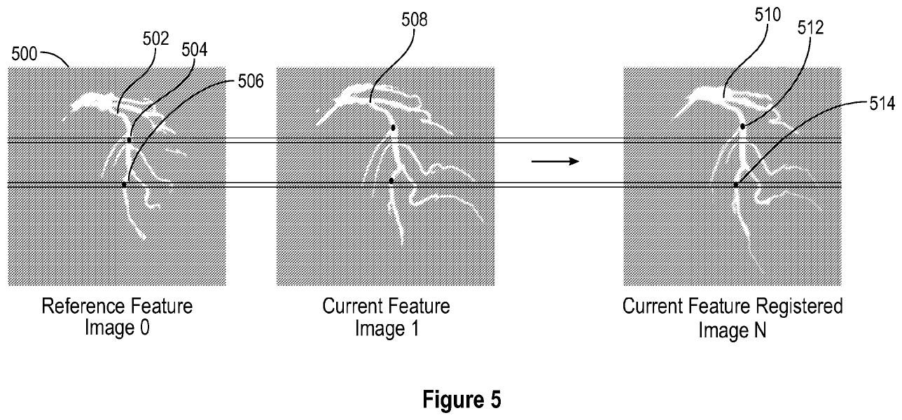

FIG. 5 shows a set of exemplary illustrations 500 for a Non-Rigid registration/generating the image deformation map to overcome motion during an imaging session and generate an accurate imaging path, according to exemplary embodiments of the present disclosure. According to certain exemplary embodiments of the present disclosure, extravascular image and/or feature registration can be performed in a non-rigid format. The exemplary registration can be used to account for heart motion and vessel movement along with vessel foreshortening to create an accurate image path in each heart/vessel position. For example, the registration can be performed by measuring a deformation map between the vasculature segmentations in a sequential images. That deformation map can then be applied to update the relative position of a detected radiopaque marker, and the sum of performing that operation on every image allows the creation of a more accurate imaging path measurement, on each extravascular image/vascular position.

Indeed, for example, vessel foreshortening can be accounted for by measuring the distance between automatically detected radiopaque marker positions in sequential extravascular images (e.g., where marker translation is not perfectly linear in distance); however, this can likely be achieved after extravascular image registration. The registration can be intensity-based, segmentation based, landmark based and/or any combination, and can be rigid (rotate/translate), or it could be affine (e.g., rigid+scaling and/or “shearing” or “skewing”), or it could be non-rigid (e.g., deformable, for example, affine along with a “vector” field) along with many other methods. The exemplary registration can be performed between different modalities (e.g., intravascular to extravascular imaging) or a single modality (in the case of an extravascular imaging set).

For example, when registering, there is a reference space (Image 0 of FIG. 5) and a current image space (Image 1 of FIG. 5). In order to register Image 1 to Image 0, there certain optimization routine exists based on the methods described above. In some exemplary cases, it is possible to utilize a machine learning model and/or a deep learning model (e.g., a convolutional neural network model, a transformer model, VoxelMorph, SynthMorph, HyperMorph, etc.) to measure and/or detect the deformation map that describes the transformation (e.g., registration, alignment, rigid registration, non-rigid registration, affine registration, non-parametric registration, etc.) from one image space to another. In some exemplary cases, it is possible to register an image with another form either imaging modality using conditional deformable templates based at least partially on convolutional networks (e.g., coronary atlas templates). In other exemplary cases, it is possible to register an image with another, from either imaging modality, using a learning procedure (e.g., supervised learning, unsupervised learning, etc.) based on diffeomorphic transforms (e.g., probabilistic diffeomorphic registration). In further exemplary cases, it is possible to register an image with another form either imaging modality using region-specific diffeomorphic Metric mapping.

Turning to the items identified in FIG. 5, within the image labelled “Reference Feature” (or Image 0), item 502 is an automatically detected vasculature feature, item 504 is a first automatically detected radiopaque marker feature (e.g., imaging position marker), and item 506 is a second automatically detected radiopaque marker feature (e.g., catheter tip position marker). Within the image labelled “Current Feature” (or Image 1), item 508 displays the automatically detected vasculature feature at a different timepoint (e.g., after movement). Within the image labelled “Current Feature Registered” (or Image N), item 510 displays the automatically detected vasculature feature 508 registered to the vascular feature 502. The registration can be performed based on any of the described learned, or traditional, registration methods described herein. Importantly, a registration/deformation map can be inferred based on e.g., an automatically detected vascular feature or intravascular device, but be applied to other automatically detected features and/or to, for example, the raw images that the feature was detected within. Such exemplary method of applying a registration map inferred from vascular features but applied to other features can also be utilized within the image labelled “Current Feature Registered” (or Image N), whereas items 512 and 514, which represent automatically detected radiopaque markers not used to register the reference frame and the current frame, have had a registration transformation applied to them, for example, to make a more accurate imaging path estimate e.g., to assess and account for vessel foreshortening.

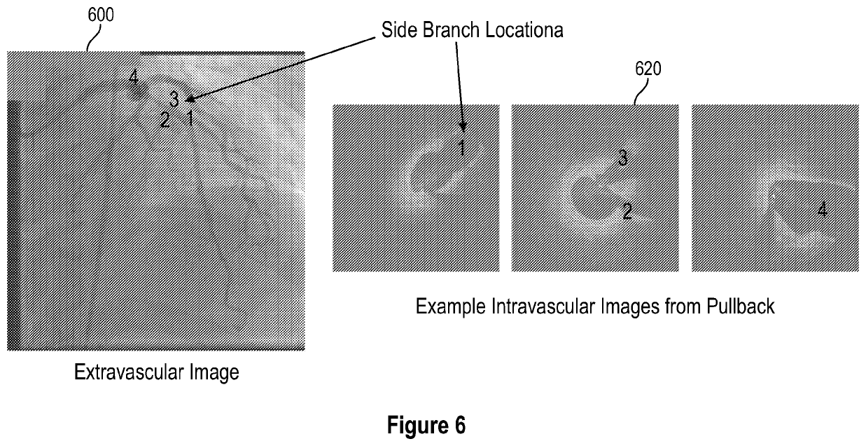

FIG. 6 shows a set of illustrations 600, 620 providing examples of Intravascular to extravascular registration refinement using detected side branch positions within the extravascular images and the intravascular images, according to exemplary embodiments of the present disclosure. While the imaging path can be created using the trajectory of the radiopaque marker during a pullback, over the registered extravascular images/features, FIG. 6 illustrates that other image features can be used to increase the longitudinal accuracy of the multi-modal (intravascular to extravascular) registration using automatically detected features in both the extravascular and intravascular images (e.g., as shown in FIG. 6, side branch positions). In some exemplary embodiments of the present disclosure, any automatically detected object and/or automatically measured attribute from either both imaging modalities independently and/or through the combined use of the disparate modalities together can be used to refine registration (e.g., stents, stenosis, lumen profile, FFR gradient, etc.).

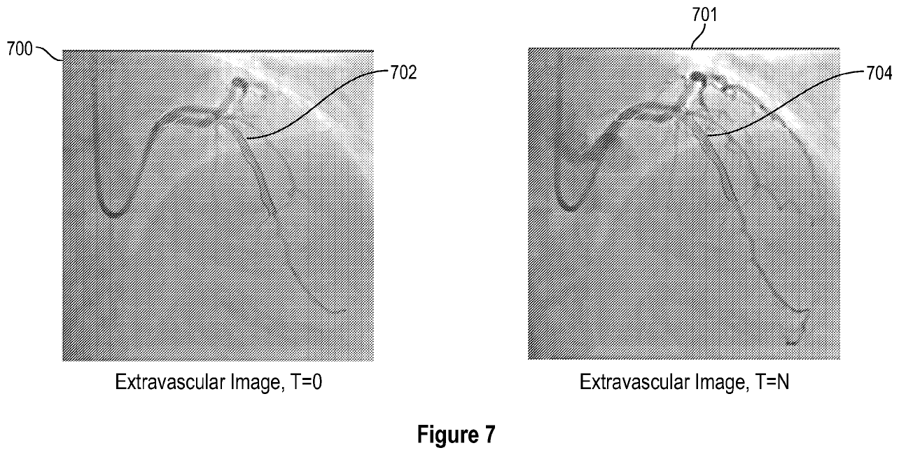

FIG. 7 shows a set of illustrations 700, 701 providing examples of Measurement profiles from the extravascular images overlayed on multiple frames from the extravascular image data set, according to exemplary embodiments of the present disclosure. As illustrated in FIG. 7, it is possible to visualize the measurements (e.g., lumen profile, external elastic membrane “EEM” profile) from the intravascular images. Turning to the items identified in FIG. 7, item 702 is a Detected Image path centerline and EEM profile at timepoint 1, color coded by pullback distance (e.g., Distal to proximal endpoints of an imaging pullback), and item 704 is a Detected Image path centerline and EEM profile at timepoint 2, color coded by pullback distance (e.g., Distal to proximal endpoints of an imaging pullback)

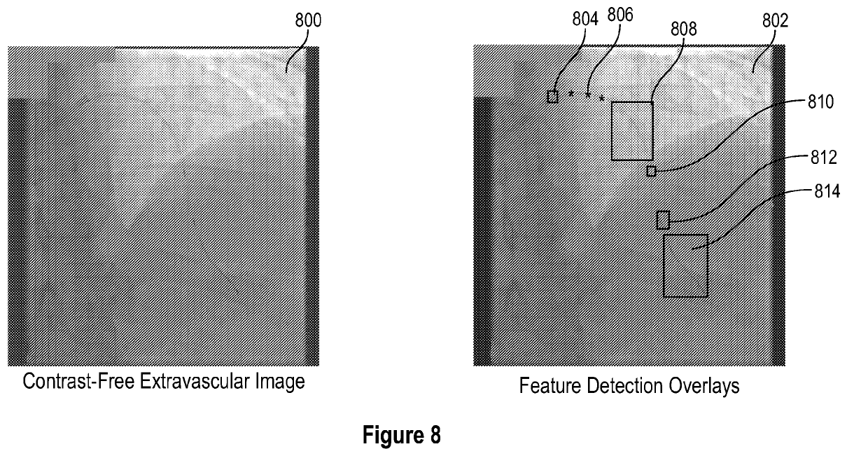

FIG. 8 shows a set of illustrations providing exemplary Contrast Free Object Detection and segmentation for Image path reconstruction and registration, according to exemplary embodiments of the present disclosure. According to certain exemplary embodiments of the present disclosure, as illustrated in FIG. 8, foreign objects (e.g., a guide catheter, a guide wire, etc.) can be detected to guide a registration of the multiple extravascular images to one another or to guide the registration of extravascular images to intravascular images. For example, to account for foreshortening or motion, an imaging path can be assumed to travel along a guidewire, and radiopaque marker positions (e.g., markers that are translated, markers that are not translated, etc.) can be used to interpolate the imaging path between extravascular frames. In other exemplary embodiments of the present disclosure, the radiopaque marker path (e.g., created by being detected in multiple extravascular images), can be enough to create an image path for each image using dynamic features located within the images (e.g., the direction of the radiopaque markers movement, the slope of the radiopaque markers, based on its angle and/or its eccentricity, etc.).

Exemplary object detection can be performed using hand-crafted procedures, such as, e.g., thresholding, morphological operations, Fourier domain analysis, edge detection, etc. as well as using machine learning or deep learning using procedures such as YOLO, for example. Turning to the items identified in FIG. 8, item 800 is an exemplary Extravascular image (e.g., a Coronary angiogram image). Some patients cannot tolerate contrast injections, in these cases, an intravascular imaging dataset (e.g., a pullback) can be registered to extravascular images without a coronary tree filled with contrast. Item 802 in FIG. 8 provides exemplary object detection Overlay examples for contrast-free, or contrast-reduced registration (e.g., registration of the multiple extravascular images to one another or to guide the registration of extravascular images to intravascular images). Item 804 indicates an automatically-detected guide catheter tip (e.g., via machine learning/deep learning procedure, for example, YOLO). Item 806 is an automatically-detected guide wire positions (e.g., via machine learning/deep learning procedure, for example, YOLO). Item 808 is an automatically-detected stent (e.g., via machine learning/deep learning procedure, for example, YOLO). Item 810 is an automatically-detected intravascular imaging catheter radiopaque marker 1 (e.g., marking a distal optics location, a radiopaque housing, obtained via machine learning/deep learning procedure, for example, YOLO, etc.). Item 812 is an automatically-detected intravascular imaging catheter radiopaque marker 2 (e.g., marking a distal position of the catheter sheath, a radiopaque marker that is not translated during an imaging pullback, obtained via machine learning/deep learning procedure, for example YOLO, etc. Item 814 is an automatically detected portion of a guidewire (e.g. marking the distal position of a blood vessel, obtained via machine learning/deep learning procedure, for example, YOLO, etc.)

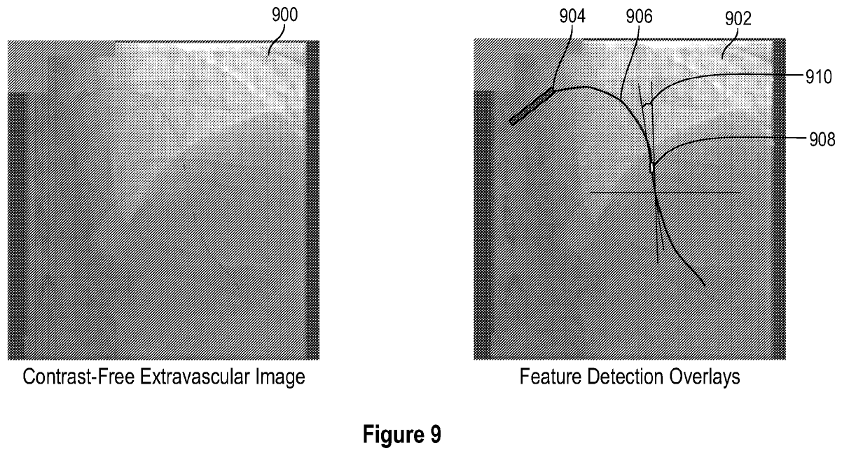

FIG. 9 shows a set of illustrations providing exemplary Contrast Free Object segmentation for image path reconstruction and registration, according to another exemplary embodiments of the present disclosure. According to certain exemplary embodiments of the present disclosure, foreign objects (e.g., a guide catheter, a guide wire, etc.) can be semantically segmented to guide a registration of the multiple extravascular images to one another or to guide the registration of extravascular images to intravascular images. For example, to account for foreshortening or motion, an imaging path can be assumed to travel along a guidewire, and radiopaque marker positions (e.g., markers that are translated, markers that are not translated, etc.) can be used to interpolate the imaging path between extravascular frames. For example, the radiopaque marker path (e.g., created by being detected in multiple extravascular images), can be enough to create an image path for each image using dynamic features located within the images (e.g., the direction of the radiopaque markers movement, the slope of the radiopaque markers, based on its angle and/or its eccentricity, etc.).

The exemplary object segmentation can be performed using hand-crafted procedures such as those based on intensity, frequency or morphological characteristics or it can be performed using machine learning or deep learning using procedures, such as U-net or Res-Net architectures, for example. Turning to the items identified in FIG. 9, item 900 is an exemplary Extravascular image (e.g., a coronary angiogram image). Some patients cannot tolerate contrast injections, in these cases, an intravascular imaging dataset (e.g., a pullback) can be registered to extravascular images without a coronary tree filled with contrast. Item 902 points to object detection Overlay examples for contrast-free, or contrast-reduced registration (e.g., registration of the multiple extravascular images to one another or to guide the registration of extravascular images to intravascular images). Item 904 is an automatically-segmented guide catheter (e.g., via machine learning/deep learning procedure, for example, U-Net, Res-Net). Item 906 is an automatically-segmented guide wire (e.g., via machine learning/deep learning procedure, for example, U-Net, Res-Net). Item 908 is an automatically-segmented radiopaque marker (e.g., via machine learning/deep learning procedure, for example, U-Net, Res-Net). Item 910 is an automatically-measured trajectory angle of the radiopaque marker.

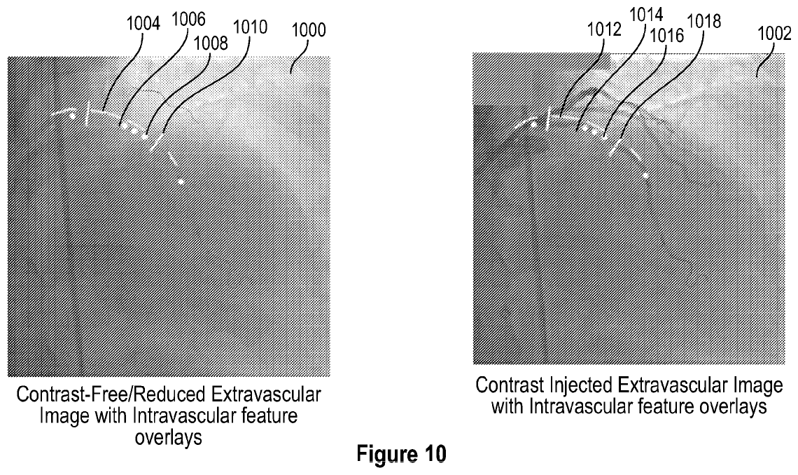

FIG. 10 shows a set of illustrations providing examples of Intravascular image feature (e.g., automatically detected features) overlayed on an extravascular image with and without contrast agents, according to exemplary embodiments of the present disclosure. Turning to the items identified in FIG. 10, item 1000 is an extravascular image without contrast injection, with automated registration or automated intravascular features overlayed. Item 1002 is an extravascular image with contrast injection, with automated registration or automated intravascular features overlayed. Item 1004 is an overlayed lipid burden (e.g. from an automatically detected lipid burden map, for example, detected using NIRS or Intravascular OCT). Item 1006 illustrates an overlayed calcium burden (e.g. from an automatically detected calcium burden map, for example, detected using NIRS or Intravascular OCT, or intravascular IVUS). Item 1008 indicates overlayed side-branch positions (e.g. detected via intravascular imaging, fore example, NIRS or Intravascular OCT, or intravascular IVUS). Item 1010 indicates overlayed stent landing flag positions (e.g., for pre-percutaneous coronary intervention (PCI) guidance on where an optimal landing zone for a stent would be). For example, these flags can be automatically generated but can be editable based on a user input (e.g. recommended based on intravascular imaging, NIRS or Intravascular OCT, or intravascular IVUS, etc.). Item 1012 provides an overlayed lipid burden (e.g. from an automatically detected lipid burden map, for example, detected using NIRS or Intravascular OCT). Item 1014 provides the overlayed calcium burden (e.g. from an automatically detected calcium burden map, for example, detected using NIRS or Intravascular OCT, or intravascular IVUS). Item 1016 provides overlayed side-branch positions (e.g. detected via either extravascular imaging, intravascular imaging or both). Item 1018 provides overlayed stent landing flag positions (e.g., for pre-PCI guidance on where an optimal landing zone for a stent would be). For example, these flags can be automatically generated but can be editable based on a user input (e.g. which can be recommended based on intravascular imaging e.g., NIRS or Intravascular OCT, or intravascular IVUS).

In some exemplary embodiments of the present disclosure, the automatic registration between intravascular and/or extravascular data as is described herein can be used, for example, as a pre-processing step to improve the determination of a physiology index (e.g., an ischemia index, an FFR index, an IMR index, a CFR index, an iFR index, etc.) based on e.g., an image-derived physiology procedure (e.g., an image-derived physiology procedure, an optimized image-derived physiology workflow, etc.). In some exemplary embodiments of the present disclosure, an image-derived physiology measurement can be registered and overlayed within an extravascular image, intravascular image or a longitudinal representation displayed within a user interface (UI).

According to further exemplary embodiments of the present disclosure, image derived physiology measurements (e.g., angiography-derived physiology measurements, OCT-derived physiology measurements, etc.) from at least one of the various imaging modalities can be used to improve (e.g., refine registration). For example, the position of lowest OCT-derived FFR along an OCT imaging pullback can be the point on an angiography image with the smallest lumen diameter, and this information can improve the registration between the OCT and Angiography modalities. Similarly, for example, the position of lowest OCT-derived FFR along an OCT imaging pullback can be or include the position of lowest angiography-derived FFR, and this information can improve the registration (e.g., automatic registration). between the OCT and Angiography modalities According to an exemplary embodiment of the present disclosure, the automated image path registration can be performed during a pre-PCI procedure or during a post-PCI procedure. During a post-PCI procedure, more than one intravascular imaging datasets and more than one extravascular datasets (e.g., a pre-PCI dataset and a post-PCI dataset) can be further automatically registered, using the strategies described herein, to compare a pre-and post-treatment measurement (e.g., pre-and post-treatment lumen profiles). According to some exemplary embodiments of the present disclosure, a pre-PCI intravascular imaging data can be registered to both a pre-PCI extravascular imaging dataset as well as a-PCI extravascular imaging dataset. According to some exemplary embodiments of the present disclosure, the a pre-PCI intravascular imaging data can be registered to a post-PCI intravascular imaging dataset (e.g., and an extravascular imaging dataset can optionally be used to aid in the registration). Information derived from such exemplary embodiments can include treatment confidence assessments, risk assessments (patient level risk or plaque level risk), malapposition, treatment accuracy (e.g., stent placement accuracy) among other important outcome indices (e.g., functional outcome indices).

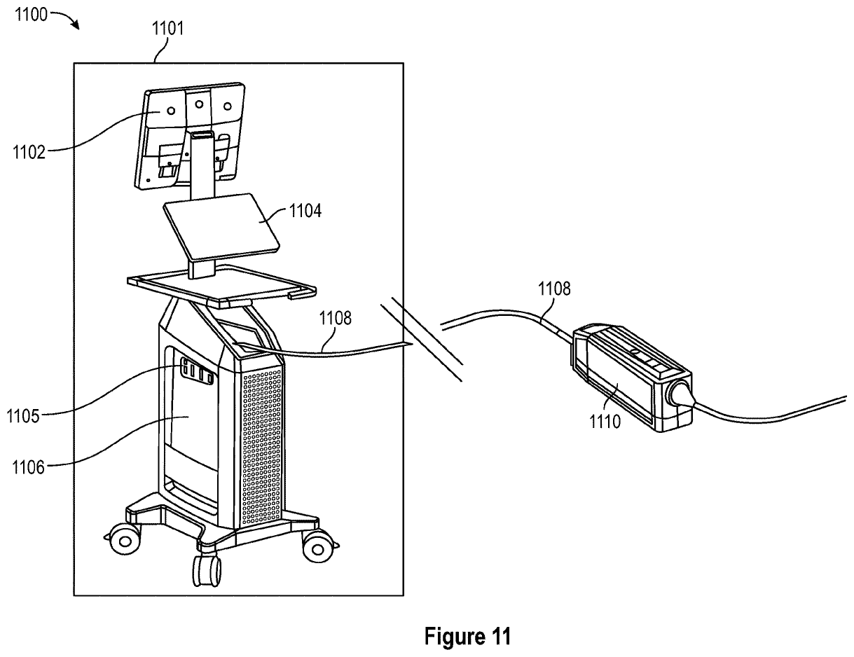

In some exemplary embodiments of the present disclosure, an automated detection algorithm can infer which coronary segment (e.g., from an agreed upon coronary atlas or description, a coronary artery anomalies “CAAs” defined segment, etc.) and/or coronary vessel corresponds (e.g., LAD, RCA, LCx, left main, etc.) to another automatically detected feature or a position within an image. In some exemplary embodiments, an automatically detected (e.g., segmented) coronary features can be first registered to a coronary atlas (e.g., a general coronary atlas, a personalized coronary atlas, a 3D coronary atlas, a 2D coronary atlas, etc.). In some exemplary embodiments of the present disclosure, an automatically detected (e.g., segmented) coronary feature can be first registered to a 3D dataset such as one derived from a coronary computed tomography angiography (CTA/CCTA) dataset. In some exemplary embodiments of the present disclosure, a previously acquired CTA dataset can assist with the automated registration of synchronized intravascular and/or extravascular imaging data. FIG. 11 is a system diagram of an intravascular imaging system 1101, according to an exemplary embodiment of the present disclosure. For example, an imaging system 1101 can comprise a display configured to acquire intravascular data and register the intravascular data with extravascular data. In some exemplary instances, the intravascular data can comprise one or more intravascular images of a blood vessel. In some exemplary cases, the extravascular data can comprise one or more extravascular images (e.g., x-ray angiogram, MRI, etc.) of blood vessel shape, physiology, anatomy, or any combination thereof. In some exemplary cases, the imaging system can comprise a computer system (1106, 1410) to process intravascular, extravascular, user interaction, or any combination thereof data.

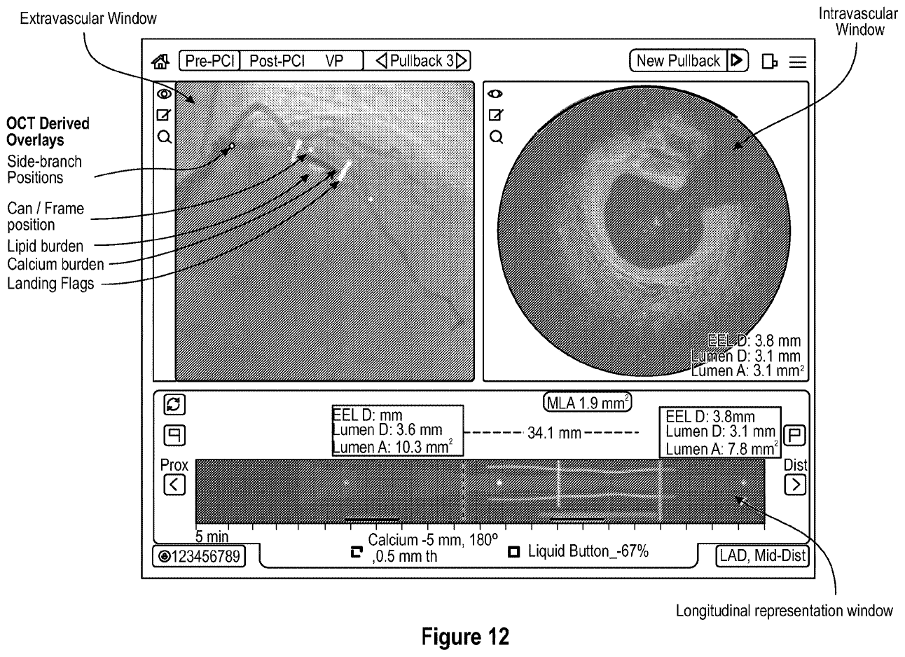

The user interaction data can comprise a user inputting data into the imaging system 1101, where the data can comprise patient information, landmark designation, selecting system operation modes, image processing functions, or any combination thereof. In some exemplary cases, a user can input data into the imaging system 1101 with a mouse and/or keyboard electrically coupled with the computer system (1106, 1410). A user can visualize a view configured (i.e., user interface) to input data into the system via a first monitor 1102 and/or a second monitor 1104. In some exemplary cases, the first monitor 1102 and/or the second monitor 1104 can comprise a touchscreen interface and key board for interacting, acquiring, or any combination thereof actions conducted on the intravascular and/or extravascular data. In some exemplary cases, the user interaction data can comprise data resulting from a user interacting with the extravascular and/or intravascular data (e.g., rotating, zooming in, adjusting contrast, adjusting brightness, measuring a distance, etc.). The computer system (1106, 1410) can include or be in communication with an electronic display 1414 (e.g., the first monitor 1102 and/or the second monitor 1144) that can comprise one or more view configurations (i.e., user interface (UI)) 1416, as also shown in FIG. 12, described elsewhere herein, for viewing the intravascular data, extravascular data, a registered and/or combination of the intravascular and extravascular data, or any combination thereof.

In some exemplary cases, the computer system (1106, 1410) can comprise an input interface 1105, where the input interface 1105 can comprise one or more input points and/or ports electrically coupled with the computer system (1106, 1410). The input interface 1105 can receive one or more data and/or streams of data from one or more imaging systems. For example, the input interface 1105 can receive an x-ray angiography data, where the computer system (1106, 1410) can then register the x-ray angiography data with the intravascular data. In some exemplary cases, the input interface 1105 can receive angiography-derived physiology, MRI, computed tomography, spatial positional, intravascular sensor (e.g., intravascular physiology), or any combination thereof data from one or more medical devices to be displayed and/or registered to the intravascular data. In some exemplary instances, the input interface 105, can receive the data to register to the extravascular data, as described elsewhere herein, wirelessly through an ad-hoc WIFI, Bluetooth, radiofrequency, or any combination thereof wireless communication platform.

In some exemplary cases, the computer system (1106, 1410) can process data with one or more processors 1404, described elsewhere herein. In some exemplary instances, the one or more processors can comprise processors of one or more graphical processing units, integrated circuit, or any combination thereof processors. The graphical processing units provide the configuration to process complex large datasets due to their highly parallel processor architecture. For example, processing data with one or more graphical processing units can provide the exemplary system with the capability of registering the intravascular data with a real-time stream of extravascular data.

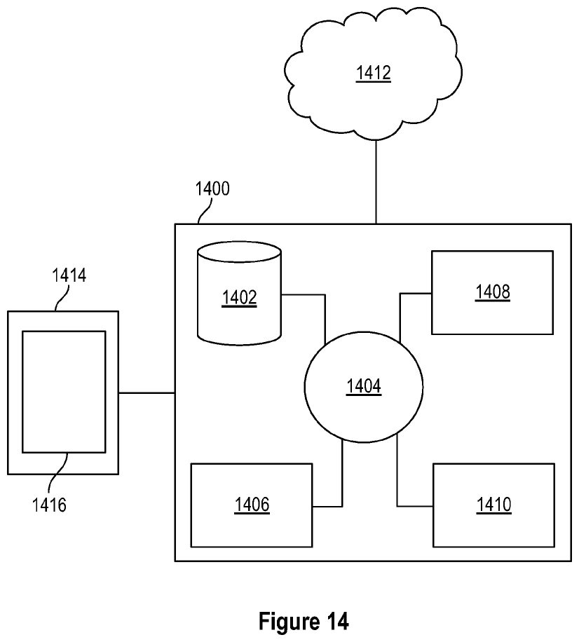

In some exemplary cases, the computer system (1106, 1410) can be configured to process the intravascular and extravascular data and/or images. The computer system (1400, 1106) as shown in FIG. 14, can comprise a central processing unit and/or graphical processing (CPU and/or GPU, also “processor” and “computer processor” herein) 1404, which can be a single core or multi core processor, or a plurality of processor for parallel processing. The computer system (1106, 1410) can further comprise memory or memory locations 1406 (e.g., random-access memory, read-only memory, flash memory), electronic storage unit 1402 (e.g., hard disk), communications interface 1408 (e.g., network adapter) for communicating with one or more other devices, and peripheral devices 1410, such as, e.g., cache, other memory, data storage and/or electronic display adapters. The memory 1406, storage unit 1402, communications interface 1408, and peripheral devices (e.g., mouse, keyboard, etc.) 1410 can be in communication with the CPU and/or GPU 1404 through a communication bus (solid lines), such as a motherboard. The storage unit 1402 can be a data storage unit (or a data repository) for storing data. The computer system (1106, 1410) can be operatively coupled to a computer network (“network”) 1412 with the aid of the communication interface 1408. The network 1412 can be the Internet, an internet and/or extranet, or an intranet and/or extranet that is in communication with the Internet. The network 1412 may, in some exemplary cases, be a telecommunication and/or data network. The network 1412 can include one or more computer servers, which can facilitate distributed computing, such as cloud computing. The network 1412, in some exemplary cases with the aid of the computer system (1106, 1410), can implement a peer-to-peer network, which can facilitate devices coupled to the computer system (1106, 1410) to behave as a client or a server.

The CPU and/or GPU 1404 can execute a sequence of machine-readable instructions, which can be embodied in a program or software. The instructions can be directed to the CPU and/or GPU 1404, which can subsequently program or otherwise configured the CPU/GPU 1404 to acquire data and/or process data produced by the imaging system described elsewhere herein.

In some exemplary embodiments of the present disclosure, the computer system (1106, 1410) central processing unit and/or graphical processing unit 1404 can execute machine executable or machine-readable code that can be provided in the form of software to transfer data generated by the imaging system to a network and/or cloud 1412 for further processing, classification, data clustering, or any combination thereof operations. In some exemplary instances, the data can comprise the intravascular and/or extravascular data, described elsewhere herein. In some exemplary cases, the data can comprise image pixel data. In some exemplary instances, the pixel data can comprise optical coherence tomography, x-ray angiography, computed tomography, intravascular ultrasound, spectroscopy, MRI, or any combination thereof image pixel data.

In some embodiments, the CPU and/or GPU 1104 can be part of a circuit, such as an integrated circuit. One or more other components of the system 1410 can be included in the circuit. In some exemplary cases, the circuit can comprise an application specific integrated circuit (ASIC).

The storage unit 1402 can store files, such as drivers, libraries, and saved programs. The storage unit 1402 can store acquired x-ray angiography, optical coherence tomography, intravascular ultrasound, near infrared spectroscopy, photoacoustic or any combination thereof data and/or images. In some exemplary cases, the intravascular and/or extravascular data and/or images can be stored in the cloud, a medical system electronic medical records (e.g., EPIC), or any combination thereof locations. The computer system (1106, 1410), in some exemplary cases, can comprise one or more additional data storage units that are external to the computer system (1106, 1410), such as, located on a remote server that is in communication with the computer system (1106, 1110) through an intranet or the internet 1412.

In some exemplary cases, the imaging system 1101 can be in electrical and/or optical communication to an imaging probe actuator 1110, and an imaging probe 1112, as seen in The imaging system 1101 can be in electrical and/or optical communication with the imaging probe actuator 1110 through one or more electrical and/or optical communication wires 1108. In some exemplary cases, the imaging probe 1112 can be releasably coupled to the imaging probe actuator 110, such that a first imaging probe can be removed from the imaging probe actuator and replaced with a second imaging probe.

In some exemplary instances, the imaging probe can comprise an intravascular imaging probe. The intravascular imaging probe can comprise an optical coherence tomography, intravascular ultrasound, reflectance, photoacoustic, near infrared spectroscopy, fluorescence, or any combination thereof imaging probes. In some exemplary instances, the imaging probe can obtain, collected, and/or detect intravascular data from an inner lumen and/or body of a blood vessel. In some exemplary cases, the intravascular data can comprise two-dimensional (e.g., circular cross-sectional data), and/or volumetric intravascular data (i.e., one or more two-dimensional circular cross-sectioned data as a function of the length of the optical axis of the imaging probe). In some exemplary cases, the imaging probe can comprise one or more radio-opaque markers and/or indicia that can be visualized on extravascular imaging modalities e.g., x-ray angiography, computed tomography, MRI, or any combination thereof extravascular imaging modalities.

In some exemplary instances, the imaging probe actuator 1110 can rotate and/or translate the imaging probe 1112, to obtain two and/or three-dimensional intravascular datasets. In some exemplary cases, the probe can be rotated by a stepper motor, dc-brushless motor, or any combination thereof motors coupled to an optic rotary joint. In some exemplary cases, the imaging probe actuator 1110 can translate the imaging probe 1112 with a stage, where the stage can comprise a linear and/or a planar translational stage. The stage translation and the rotation of the imaging probe actuator 1110 can be set and/or adjusted by a user via the one or more interfaces of the imaging system 1101, described elsewhere herein. In some exemplary instances, the stage translation and the rotation of the imaging probe actuator 1110 can be determine and/or set by the system based on pre-set standard values for a particular type of imaging procedure or frequently used settings.

Exemplary aspects of the systems and methods provided herein, such as the computer system (1106, 1410), can be embodied in programming. Various aspects of the technology can be thought of a “product” or “articles of manufacture” typically in the form of a machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of a computer, processor the like, or associated modules thereof, such as various semiconductor memories, tape drives, disk drives and the like, which can provide non-transitory storage at any time for the software program. All or portions of the software can at times be communicated through the Internet or various other telecommunication networks. Such communications, for example, can facilitate loading of the software from one computer or processor into another, for example, from a management server or host computer into the computer platform of an application server. Thus, another type of media that can bear the software elements includes optical, electrical, and electromagnetic waves, such as used across physical interfaces between local devices, through wired and optical landline networks and over various air-links. The physical elements that carry such waves, such as wired or wireless links, optical links, or the like, also can be considered as media bearing the software. As used herein, unless restricted to non-transitory, tangible “storage” media, term such as computer or machine “readable medium” refer to any medium that participates in providing instructions to a processor for execution.

Thus, a machine readable medium, such as computer-executable code, can take many forms, including but not limited to, a tangible storage medium, a carrier wave medium or physical transmission medium. Non-volatile storage media can include, for example, optical or magnetic disks, such as any of the storage devices in any computer(s) or the like, such as can be used to implement the databases, etc. Volatile storage media can include dynamic memory, such as main memory of such a computer platform. Tangible transmission media includes coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefor include for example: a floppy disk, a flexible disk, hard disk, magnetic tape, any other magnetic medium, a CD-ROM, DVD or DVD-ROM, any other optical medium, punch cards paper tape, any other physical storage medium with pattern of holes, a RAM, a ROM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media can be involved in carrying one or more sequences of one or more instruction to a processor for execution.

### Computer Systems and Machine Learning Models

In some embodiments, the system 100 disclosed herein can comprise a computer system (1106, 1410) suitable for implementing machine learning procedures and/or predictive models configured to analyze, process, segment and/or label extravascular and/or intravascular data collected by the imaging system 1101, imaging probe 1112, and imaging probe actuator 1110 described elsewhere herein. In some exemplary cases, one or more intravascular and/or extravascular images can be generated from the intravascular and/or extravascular data. In some exemplary cases, predictive models e.g., machine learning models and/or machine learning procedures can analyze, extract, condense, reduce, predict, process, classify, segment or any combination thereof operations conducted on the intravascular and/or extravascular data. In some embodiments, the systems disclosed herein can implement one or more machine learning procedures and/or model(s) to identify, classify, process and/or segment regions of interest of intravascular and/or extravascular data.

In some exemplary instances, the one or more categories and/or features of the data can be provided to one or more treatment parameter machine learning model and/or procedures to determine suggested treatment and/or treatment parameters (e.g., what type of stent to place and where spatially to best place the stent to achieve clinical efficacy of treatment). The one or more treatment parameter machine learning models can be trained with prior features and corresponding treatment efficacy (i.e., whether any complications ensued after clinical intervention with the system) to generate one or more trained treatment parameter machine learning models to predict efficacious treatments. The spatial orientation of labeled features and their relationship to one another can be other features determined and considered by the treatment parameter machine learning models.

In some exemplary cases, the one or more categories and/or features of data for extravascular data can comprise background data, healthy blood vessel morphology, stenotic blood vessel morphology, or occluded blood vessel. In some exemplary cases, the one or more categories of data for the intravascular data can comprise blood vessel tissue of the epithelium, blood vessel tissue of the intima, blood vessel tissue of the adventitia, plaque within the blood vessel tissue, vulnerable plaque within the blood vessel tissue, or any combination thereof. In some exemplary cases, the one or more categories and/or features of intravascular data can comprise spectroscopic (e.g., in the near infrared) signature of the intravascular blood vessel tissue. For example, the one or more categories and/or features can classify the composition of plaque of the blood vessel based on its spectroscopic signature. In some exemplary cases, the one or more categories can comprise a calcium or a lipid spectroscopic signature. In some exemplary instances, the machine learning model and/or procedures can be used to pre-process the intravascular and/or extravascular data prior to classifying a feature of the data. In some exemplary instances, prep-processing the intravascular and/or extravascular data can comprise de-noising, smoothening, averaging, sharpening, brightness and/or contrast adjustment, or any combination thereof mathematical manipulation of the data. In some exemplary cases, the features and/or categories of the intravascular and/or extravascular data can be extracted without a pre-processing step.

In some exemplary cases, machine learning procedures can need to extract and draw relationships between features as conventional statistical techniques can not be sufficient. In some exemplary cases, machine learning procedures can be used in conjunction with conventional statistical techniques. In some exemplary cases, conventional statistical techniques can provide the machine learning procedure with pre-processed features.

In some exemplary embodiments of the present disclosure, any number of features can be classified by the machine learning procedure. The machine learning procedure can classify at least 1 feature. In some exemplary cases, the plurality of features can include between about 1 feature to 5 features. In some exemplary cases, the plurality of features can include between about 5 features to 10 features. In some exemplary cases, the plurality of features can include between about 10 features to 50 features.

In some exemplary embodiments of the present disclosure, the machine learning procedure can be, for example, an unsupervised learning procedure, supervised learning procedure, or a combination thereof. The unsupervised learning procedure can be, for example, clustering, hierarchical clustering, k-means, mixture models, DBSCAN, OPTICS procedure, VoxelMorph procedure, anomaly detection, local outlier factor, neural networks, autoencoders, deep belief nets, hebbian learning, generative adversarial networks, self-organizing map, expectation-maximization procedure (EM), method of moments, blind signal separation techniques, principal component analysis, independent component analysis, non-negative matrix factorization, singular value decomposition, or a combination thereof. The supervised learning procedure can be, for example, support vector machines, linear regression, logistic regression, linear discriminant analysis, decision trees, k-nearest neighbor procedure, neural networks, similarity learning, or a combination thereof. In some embodiments, the machine learning procedure can comprise a deep neural network (DNN). The deep neural network can comprise a convolutional neural network (CNN). The CNN can be, for example, U-Net, ImageNet, LeNet-5, AlexNet, ZFNet, GoogleNet, VGGNet, ResNet18 or ResNet, etc. Other neural networks can be, for example, deep feed forward neural network, recurrent neural network, LSTM (Long Short Term Memory), GRU (Gated Recurrent Unit), Auto Encoder, variational autoencoder, adversarial autoencoder, denoising auto encoder, sparse auto encoder, Boltzmann machine, RBM (Restricted BM), deep belief network, generative adversarial network (GAN), deep residual network, capsule network, or attention/transformer networks, etc.

In some exemplary instances, the machine learning model can comprise clustering, scalar vector machines, kernel SVM, linear discriminant analysis, Quadratic discriminant analysis, neighborhood component analysis, manifold learning, convolutional neural networks, reinforcement learning, random forest, Naive Bayes, gaussian mixtures, Hidden Markov model, Monte Carlo, restrict Boltzmann machine, linear regression, or any combination thereof.

In some exemplary cases, the machine learning procedure can include ensemble learning procedures such as bagging, boosting and stacking. The machine learning procedure can be individually applied to the plurality of features extracted.

In some exemplary embodiments, the systems can apply one or more machine learning procedures and/or an ensemble of machine learning procedures.

In some embodiments, the machine learning procedure can have a variety of parameters. The variety of parameters can be, for example, learning rate, minibatch size, number of epochs to train for, momentum, learning weight decay, or neural network layers etc.

In some exemplary embodiments, the learning rate can be between about 0.00001 to 0.1.

In some embodiments, the minibatch size can be at between about 16 to 128.

In some exemplary embodiments, the neural network can comprise neural network layers. The neural network can have at least about 2 to 1000 or more neural network layers.

In some exemplary embodiments, the number of epochs to train for can be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more.

In some exemplary embodiments, the momentum can be at least about 0.1, 0.2, 0.3, 0.4, 0.5, 0.6, 0.7, 0.8, 0.9 or more. In some exemplary embodiments, the momentum can be at most about 0.9, 0.8, 0.7, 0.6, 0.5, 0.4, 0.3, 0.2, 0.1, or less.

In some exemplary embodiments, learning weight decay can be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some exemplary embodiments, the learning weight decay can be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less.

In some exemplary embodiments, the machine learning procedure can use a loss function. The loss function can be, for example, regression losses, mean absolute error, mean bias error, hinge loss, Adam optimizer and/or cross entropy.

In some embodiments, the parameters of the machine learning procedure can be adjusted with the aid of a human and/or computer system.

In some embodiments, the treatment parameter machine learning model and/or procedures can prioritize certain features. The treatment parameter machine learning model and/or procedures can prioritize features that can be more relevant for determining anatomical and/or physiologic features to characterize variation in blood vessel geometry and composition. In some exemplary cases, the blood vessel geometry and composition can classify a portion of a blood vessel as diseased (e.g., thin cap fibroatheroma, vulnerable plaque, stabile plaque, etc.). In some exemplary cases, the features can be prioritized using a weighting system. In some exemplary cases, the features can be prioritized on probability statistics based on the frequency and/or quantity of occurrence of the feature. The machine learning procedure can prioritize features with the aid of a human and/or computer system.

In some exemplary embodiments, one or more of the features can be used with machine learning or conventional statistical techniques to determine if a segment of intravascular and/or extravascular data is likely to contain artifacts. The identified artifacts can be a result of optical misalignment, movement of the subject during intravascular and/or extravascular data acquisition, laser power instability, laser pulse frequency jitter, movement of the subject via breathing or micro-tremors, or any combination thereof artifact. In some exemplary cases, movement sensors or other sensors can be used as an additional input to the artifact reduction machine learning model and/or procedure. In some exemplary cases, the identified artifacts can be rejected from being used in blood vessel anatomy and/or disease classification.

In some exemplary cases, the machine learning procedure can prioritize certain features to reduce calculation costs, save processing power, save processing time, increase reliability, or decrease random access memory usage, etc.

Methods as described herein can be implemented by way of machine (e.g., computer processor) executable code stored on non-transitory electronic storage medium of the computer system 1410, such as, for example, on the memory 1406 or electronic storage unit 1402. The machine executable or machine-readable code can be provided in the form of software. During use, the code can be executed by the processor (i.e., CPU and/or GPU) 1404. In some exemplary instances, the code can be retrieved from the storage unit 1402 and stored on the memory 1406 for ready access by the processor 1404. In some exemplary instances, the electronic storage unit 1402 can be precluded, and machine-executable instructions are stored on memory 1406.

The code can be pre-compiled and configured for use with a machine having a processor adapted to execute the code or can be compiled during runtime. The code can be supplied in a programming language that can be selected to enable the code to be executed in a pre-compiled or as-compiled fashion.

FIG. 12 shows an exemplary display of co-registered Intravascular imaging and extravascular imaging on Intravascular imaging with the user interface (UI), using the exemplary system of FIG. 11, according to an exemplary embodiment of the present disclosure. As shown in FIG. 12, the UI has three primary viewports for viewing multi-modal data: a first extravascular viewport for displaying angiography data and a second intravascular viewport for displaying intravascular OCT-NIRS data as well as a third longitudinal viewport for displaying longitudinal images, measurements & representations. In this example, the extravascular viewport, displaying a single frame from an extravascular angiography dataset, comprises overlays for the automatically detected intravascular features (such as calcium burden, lipid burden and side branches locations) automatically co-registered to the vessel architecture within the extravascular image. In this example, the extravascular viewport also comprises overlays for automatically detected extravascular features, such as side-branch locations, radiopaque marker locations (e.g., a radiopaque can that designates a intravascular imaging position in time), landing flags for treatment guidance (e.g., stent landing flags, e.g., automatically generated stent landing flags, e.g., manually selected stent landing flags). The intravascular viewport shows a single frame from an intravascular dataset (e.g., multi-modal OCT-NIRS data), comprising overlays for automatically detected intravascular features such as plaque composition. Plaque composition (e.g., lipid presence, e.g., lipid concentration) can be automatically detected by a spectroscopy modality (e.g., NIRS or fluorescence) or an imaging modality (e.g., OCT or IVUS). The longitudinal viewport illustrates a longitudinal presentation of 1D, 2D and 3D information from either the intravascular data or extravascular data or any automatically detected and/or measure feature from either data source (e.g., lumen diameter/area, e.g., plaque size and composition, e.g., plaque size and composition above a certain clinical threshold.). Any of the viewports can also be configured to display a-priori data or patient information. Any of the viewports can also be configured to further comprise a representation of physiology measurement data (e.g., image-derived physiology, e.g., OCT-derived physiology, e.g., Angiography-derived physiology).

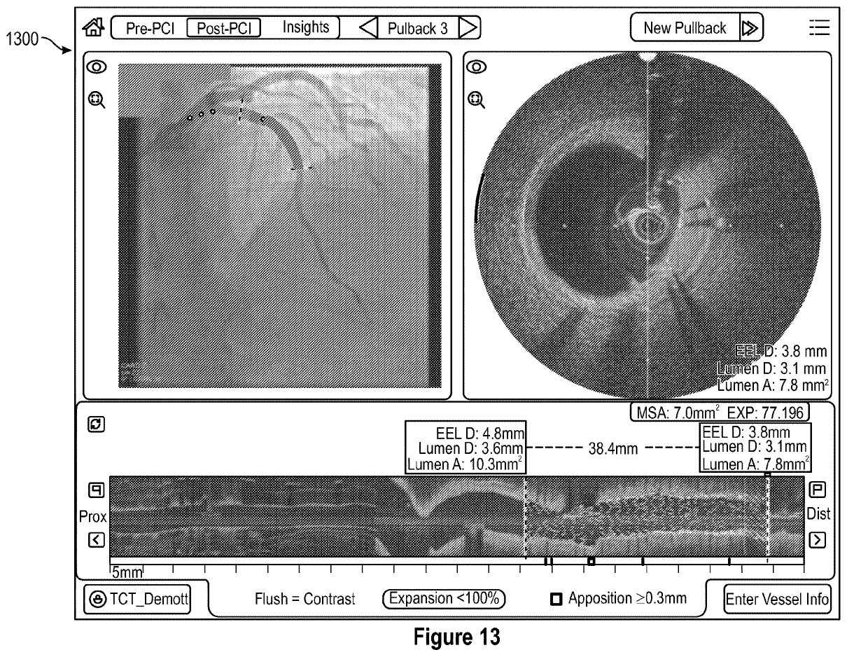

FIG. 13 illustrates an exemplary display 1300 of co-registered Intravascular imaging and extravascular imaging on Intravascular imaging with the user interface (UI), using the exemplary system of FIG. 11, according to yet another exemplary embodiment of the present disclosure. Angiography overlays can include under expansion markers and appositions markers as well as flag positions demarcating a stent position after a stent is place. All the overlays can be automatically generated from the intravascular imaging.

In particular, FIG. 14 illustrates a computer system 1410 according to an exemplary embodiment of the present disclosure. To summarize, item 1402 represents a storage unit, item 1404 represents a processor (i.e., CPU and/or GPU), item 1406 represents a random access memory (RAM), item 1408 represents a communications interface (e.g., network adapter), item 1412 represents a network, item 1414 represents an electronic display and item 1416 represents a UI.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

1. A method for obtaining information regarding at least one feature of an object, comprising:
acquiring an intravascular dataset and an extravascular dataset; automatically receiving information regarding the at least one feature within the extravascular dataset;
automatically generating an imaging path based on the received information regarding the at least one feature;
automatically registering the intravascular dataset to the imaging path to generate the registered intravascular data, and
automatically overlaying a representation of the registered intravascular data within at least one image of the extravascular dataset.

2. The method of claim 1, wherein the at least one feature includes a radiopaque marker on an intravascular probe.

3. The method of claim 2, wherein the registered intravascular dataset includes at least one intravascular sensing dataset.

4. The method of claim 3, wherein the at least one intravascular sensing dataset includes data regarding at least one of a pressure, an electric field, or flow.

5. The method of claim 2, wherein the registered intravascular dataset includes at least one intravascular imaging dataset.

6. The method of claim 5, wherein the at least one intravascular imaging dataset includes at least one of optical coherence tomography (OCT) data, intravascular ultrasound (IVUS) data, Near-infrared spectroscopy (NIRS) data, reflectance data or Fluorescence Lifetime Imaging Microscopy (FLIM) data.

7. The method of claim 1, wherein the extravascular dataset includes data for coronary angiography images.

8. The method of claim 1, wherein the overlayed representation indicates a location of a stenosis.

9. The method of claim 8. wherein the overlayed representation further indicates a type of plaque at the stenosis.

10. The method of claim 1, wherein the overlayed representation indicates a measurement output at a specific location along a coronary vessel.

11. The method of claim 10, wherein the measurement output comprises at least one of a plaque burden, a lipid burden, or a calcium burden.

12. The method of system 1, wherein the automatically detecting the feature comprises executing a machine learned procedure for an object segmentation.

13. The method of claim 12, wherein the machine learning procedure comprises at least one of U-Net or Res-Net.

14. The method of claim 1, wherein the automatically detecting the feature comprises executing a machine learned procedure for an object detection.

15. The method of claim 1 wherein the automatically generating the image path comprises automatically interpolating datapoints.

16. The method of claim 1, further comprising registering the image path between extravascular images within the extravascular dataset.

17. The method of claim 16, wherein the automatically registering the image path comprises executing a machine learned non-rigid transformation procedure between the images within the extravascular dataset.

18. The method of claim 1, further comprising automatically segmenting a coronary vasculature within the extravascular dataset.

19. The method of claim 1, further comprising segmenting data associated with a guide wire within the extravascular dataset.

20. The method of claim 1, wherein the intravascular dataset is a multimodal intravascular imaging and spectroscopy dataset.

21. A system for obtaining information regarding at least one feature of an object, comprising:
a computer-processing configuration which is configured or programmed to:
acquire an intravascular dataset and an extravascular dataset;
automatically receive information regarding the at least one feature within the extravascular dataset:
automatically generate an imaging path based on the received at least one feature;
automatically register the intravascular dataset to the imaging path to generate the registered intravascular data, and
automatically overlay a representation of the registered intravascular data within an image of the extravascular dataset.

22. The system of claim 21, wherein the at least one feature includes a radiopaque marker on an intravascular probe.

23. The system of claim 21, wherein the registered intravascular dataset includes at least one intravascular sensing dataset.

24. The system of claim 23, wherein the at least one intravascular sensing dataset includes data regarding at least one of a pressure, an electric field, or flow.

25. The system of claim 21, wherein the registered intravascular dataset includes at least one intravascular imaging dataset.

26. The system of claim 25, wherein the at least one intravascular imaging dataset includes at least one of optical coherence tomography (OCT) data, intravascular ultrasound (IVUS) data. Near-infrared spectroscopy (NIRS) data, reflectance data or Fluorescence Lifetime Imaging Microscopy (FLIM) data.

27. The system of claim 21, wherein the extravascular dataset includes data for coronary angiography images.

28. The system of claim 21, wherein the overlay ed representation indicates a location of a stenosis.

29. The system of claim 28, wherein the overlayed representation further indicates a type of plaque at the stenosis.

30. The system of claim 21, wherein the overlayed representation indicates a measurement output at a specific location along a coronary vessel.

31. The system of claim 30, wherein the measurement output comprises at least one of a plaque burden, a lipid burden, or a calcium burden.

32. The system of system 21, wherein the feature is automatically detected by executing a machine learned procedure for an object segmentation, (e.g., U-Net, Res-Net, etc.).

33. The system of claim 32, wherein the machine learning procedure comprises at least one of U-Net or Res-Net.

34. The system of claim 21, wherein the feature is automatically detected by executing a machine learned procedure for an object detection.

35. The system of claim 21, wherein the automatically generating the image path comprises automatically interpolating datapoints.

36. The system of claim 21, wherein the computer processing configuration is further configured to register the image path between extravascular images within the extravascular dataset.

37. The system of claim 36, wherein the image path is automatically registered by executing a machine learned non-rigid transformation procedure between the images within the extravascular dataset.

38. The system of claim 21, wherein the computer processing configuration is further configured to automatically segment a coronary′ vasculature within the extravascular dataset.

39. The system of claim 21, wherein the computer processing configuration is further configured to segment data associated with a guide wire within the extravascular dataset.

40. The system of claim 21, wherein the intravascular dataset is a multimodal intravascular imaging and spectroscopy dataset.

41-102. (canceled)
